(19) United States
(12) Patent Application Publication
(10) Pub. No.: US 2016/0051209 A1
(43) Pub. Date: Feb. 25, 2016

(54) RADIOLOGICAL IMAGING SYSTEM

(71) Applicant: Konica Minolta, Inc., Tokyo (JP)

(72) Inventors: ATSUSHI SUZUKI, Tokyo (JP); EMIEL VISSER, Tokyo (JP)

(21) Appl. No.: 14/831,368

(22) Filed: Aug. 20, 2015

(30) Foreign Application Priority Data
Aug. 22, 2014 (JP) 2014-168935

(51) Int. Cl. A61B 6/06; A61B 6/00

(52) U.S. Cl. CPC A61B 6/06; A61B 6/4429; A61B 6/52

(57) ABSTRACT

A radiological imaging system includes: a radiological imaging apparatus configured to generate image data in accordance with a dose of delivered radiation; and a radiation source configured to deliver radiation to the radiological imaging apparatus, wherein the radiation source is placed so that a tube axis of a rotating anode extends in a direction perpendicular to a body axis of an object being imaged.

### CROSS REFERENCE TO RELATED APPLICATIONS

The present invention claims priority under 35 U.S.C. §119 to Japanese Application No. 2014-168935 filed on Aug. 22, 2014, the entire content of which is incorporated herein by reference.

### BACKGROUND OF THE INVENTION

1. Field of the Invention

The present invention relates to a radiological imaging system, and more particularly, to a radiological imaging system that performs radiological imaging by delivering radiation from a radiation source to a radiological imaging apparatus.

2. Description of the Related Art

As a method of capturing an image of a relatively wide area of the body of a patient such as the upper or lower half of the body, so-called long-length imaging is known. In long-length imaging, a radiological imaging apparatus (a flat panel detector) captures radiological images by releasing radiation from a radiation source while changing the position thereof along the body axis of the object being imaged. Radiological images captured in long-length imaging is normally combined into a single radiological image through image processing. The three methods described below are well-known methods for such long-length imaging.

As shown in FIG. 16A, a first example method is a method of performing long-length imaging by moving a radiation source S in the direction of the body axis of an object H in synchronization with change in the position of a radiological imaging apparatus F in the direction of the body axis of the object H (in the vertical direction in FIG. 16A), and releasing radiation in respective positions. Hereinafter, this imaging method will be referred to as the parallel method, for the radiological imaging apparatus F and the radiation source S move parallel to each other.

As shown in FIG. 16B, a second example method is a known method of performing long-length imaging by changing the release direction of the radiation source S in synchronization with change in the position of the radiological imaging apparatus F in the direction of the body axis of the object H, and releasing radiation in respective release directions while swaying the head of the radiation source S without change in the position of the radiation source S. Hereinafter, this imaging method will be referred to as the tilted tubular-bulb method, for the release direction of the tubular bulb (or a rotating anode (see FIG. 3, which will be described later)) of the radiation source S is tilted, and radiation is then released.

Further, as shown in FIG. 17, a third example method is a known method of performing long-length imaging by placing a collimator C between the radiation source S and the radiological imaging apparatus F. This collimator C has an aperture (see FIG. 1B, which will be described later). The position of the aperture is changed by moving the collimator C in the direction of the body axis of the object H in synchronization with change in the position of the radiological imaging apparatus F in the direction of the body axis of the object H, without any change in the position of the radiation source S and the release direction. The position of the aperture of the collimator C is changed so that the radiation that has been released from the radiation source S and has passed through the aperture of the collimator is certainly delivered to the radiological imaging apparatus F (see JP 2013-226243 A, for example). Hereinafter, this imaging method will be referred to as the collimator method, for the collimator C is used.

FIGS. 16A and 16B and FIG. 17 illustrate cases where the radiological imaging apparatus F is moved between two positions, an upper position and a lower position, during imaging. However, it is of course possible to appropriately determine the number of positions to which the radiological imaging apparatus F can be moved in accordance with the body site being imaged, for example. Although not shown in the drawings, in a case where imaging is performed on an object in a lying state or in a recumbent position, instead of an upright position as shown in FIGS. 16A and 16B and FIG. 17, the body axis of the object extends in a horizontal direction.

By either the parallel method illustrated in FIG. 16A or the tilted tubular-bulb method illustrated in FIG. 16B, the radiation source S needs to be moved. However, it is not necessarily easy to move a heavy radiation source S at a high speed, and a mechanism for moving such a radiation source S at a high speed is normally expensive. Therefore, it is difficult to prevent an increase in the cost of an entire radiological imaging system including the radiation source S. Furthermore, if the radiation source S is designed to be movable with high precision, the cost becomes even higher.

If the radiation source S is designed to be moved not at a high speed but at a low speed, an increase in cost can be prevented, but it is very difficult to shorten the intervals between imaging operations performed by releasing radiation several times while changing the position and the release direction of the radiation source S. As a result, the time required for the long-length imaging becomes longer, and stress is put on the patient as the object.

If the accuracy in changing the position of the radiation source S and changing the release direction is sacrificed, it is necessary to keep as large combining portions (or overlap widths) on which adjacent radiological images are to be overlapped, so as to accurately combining radiological images captured by the radiological imaging apparatus F being moved.

By the collimator method illustrated in FIG. 17, on the other hand, there is no need to move the radiation source S by changing the position of the radiation source S or changing the release direction, and a lightweight collimator C is simply moved in synchronization with movement of the radiological imaging apparatus F. Accordingly, the collimator C can be moved at a high speed, and the time required for long-length imaging can be made shorter than that in the case of the parallel method or the tilted tubular-bulb method. Thus, the stress on the patient as the object can be reduced.

Also, a mechanism for accurately moving the position of the aperture of the lightweight collimator C is not very expensive, and accordingly, an increase in the cost of the entire radiological imaging system including the radiation source S and the collimator C can be prevented. Furthermore, the combining portions of radiological images captured through the long-length imaging can be made smaller, and the radiological imaging apparatus F can be moved over a wide range during the imaging. In this manner, the use of the collimator method provides many advantages.

By the collimator method, however, radiation needs to be delivered from the radiation source S to the entire moving area of the radiological imaging apparatus F that moves in the direction of the body axis of the object H, as shown in FIG. 17. That is, in a case where the radiological imaging apparatus F moves in the vertical direction as shown in FIG. 17, radiation needs to be delivered from the radiation source S to the area extending from the upper end of the radiological imaging apparatus F located in the uppermost position to the lower end of the radiological imaging apparatus F moved to the lowermost position.

By the parallel method and the tilted tubular-bulb method illustrated in FIGS. 16A and 16B, on the other hand, radiation is delivered from the radiation source S to the radiological imaging apparatus F while the position and the release direction of the radiation source S are changed in synchronization with movement of the radiological imaging apparatus F. Therefore, radiation needs to be delivered from the radiation source S only to the area equivalent to one radiological imaging apparatus F. In this case, there is no need to widen the range of radiation to be released from the radiation source S to a size as large as that in the case of the collimator method.

In radiological imaging including long-length imaging, the tube axis of the rotating anode of the radiation source S is normally placed parallel to the body axis of the patient. This is because the output intensity of radiation characteristically varies in the direction of the tube axis of the rotating anode as will be described later. That is, if the tube axis of the rotating anode of the radiation source S is placed parallel to the body axis of the patient, the output intensity of radiation varies in the direction of the tube axis of the rotating anode due to a heel effect described later, but does not vary in a direction perpendicular to the tube axis.

For a radiogram interpreter, gradation in a direction perpendicular to the body axis of the patient is often more important in diagnosis than gradation in the direction of the body axis. Therefore, the tube axis of the rotating anode of the radiation source S is normally placed parallel to the body axis of the patient.

However, if the tube axis of the rotating anode of the radiation source S is placed parallel to the body axis of the patient as described above, the intensity I_{R} of radiation released from the radiation source S is almost zero at a predetermined angle (14 degrees in FIG. 5) or greater with respect to the direction of release of radiation from the radiation source S, due to a heel effect, as will be described later with reference to FIG. 5. That is, if the tube axis of the rotating anode of the radiation source S is placed parallel to the body axis of the patient, the field of radiation to be released from the radiation source S (or the area to which radiation is delivered) becomes narrower in the direction of the tube axis or the direction of the body axis of the patient.

In such a situation, so as to realize delivery of radiation from the radiation source S to the entire moving area of the radiological imaging apparatus F moving in the direction of the body axis of the object H as in the case of the collimator method illustrated in FIG. 17, the position of the radiation source S needs to be moved further away from the radiological imaging apparatus F and the object H, as can be seen from comparisons with the parallel method and the tilted tubular-bulb method illustrated in FIGS. 16A and 16B.

That is, in a case where the above described collimator method is employed, the distance between the radiation source S and the radiological imaging apparatus F needs to be increased, the radiation source S being a conventional radiation source. Therefore, if the imaging room in which the radiation source S and others are placed is small and narrow, the collimator method cannot be employed. Such a problem occurs mainly because the field of radiation to be released from the radiation source S cannot be widened in the direction of the body axis of the object H as described above, the radiation source S being a conventional radiation source.

### SUMMARY OF THE INVENTION

The present invention has been made in view of the above problems, and an object thereof is to provide a radiological imaging system including a radiation source that can widen the radiation field in the direction of the body axis of an object, the radiation field being the field of radiation to be released from the radiation source.

To achieve the abovementioned object, according to an aspect, a radiological imaging system reflecting one aspect of the present invention comprises: a radiological imaging apparatus that generates image data in accordance with a dose of delivered radiation; and a radiation source that delivers radiation to the radiological imaging apparatus, wherein the radiation source is placed so that the tube axis of a rotating anode extends in a direction perpendicular to the body axis of an object being imaged.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1A is a schematic view of the configuration of an entire radiological imaging system according to this embodiment;

FIG. 1B is a diagram showing an example structure of a collimator;

FIG. 2A is a diagram showing an example structure of a conventional radiation source in the case of an upright position;

FIG. 2B is a diagram showing an example structure of the conventional radiation source in the case of a recumbent position;

FIG. 3 is a diagram showing the structure inside the frame of a radiation source and the tube axis of a rotating anode;

FIG. 4A is a diagram of the focal point of radiation, seen from the object side;

FIG. 4B is a diagram showing that the target surface of a rotating anode is a sloping surface;

FIG. 5 is a diagram for explaining the distribution of the intensity of radiation in a case where a heel effect occurs;

FIG. 6A is a diagram showing an example structure of a radiation source according to this embodiment in the case of an upright position;

FIG. 6B is a diagram showing an example structure of the radiation source in the case of a recumbent position;

FIG. 7 is a diagram for explaining that radiological imaging apparatuses are arranged in the direction of the body axis of an object, and long-length imaging is performed through one-time release of radiation;

FIGS. 8A through 8C are diagrams for explaining a method of switching from a conventional radiation source to a radiation source according to this embodiment;

FIG. 9 is a diagram for explaining another method of switching from a conventional radiation source to a radiation source according to this embodiment;

FIG. 10 is a diagram for explaining an example of representation of a position P with respect to a radiation source;

FIG. 11A is a diagram showing an example structure of a grid;

FIG. 11B is a cross-sectional view of the grid taken along the line X-X defined in FIG. 11A;

FIG. 12 is a flowchart showing the procedures in a specific example of an image generation process in long-length imaging;

FIG. 13A is a cross-sectional view of an example structure of an imaging stand for recumbent imaging, with a single radiological imaging apparatus being installed therein;

FIG. 13B is a cross-sectional view of an example structure of an imaging stand for recumbent imaging, with radiological imaging apparatuses being installed therein;

FIG. 14A shows an example structure of an imaging stand for upright imaging in a case where long-length imaging is performed with radiological imaging apparatuses;

FIG. 14B shows an example structure of an imaging stand for upright imaging in a case where long-length imaging is performed with a single radiological imaging apparatus;

FIG. 15 is an enlarged view of the pulley portion of the imaging stand shown in FIG. 14A;

FIG. 16A is a diagram illustrating a case where long-length imaging is performed by a parallel method;

FIG. 16B is a diagram illustrating a case where long-length imaging is performed by a tilted tubular-bulb method; and

FIG. 17 is a diagram for explaining a case where long-length imaging is performed by a collimator method using a conventional radiation source.

### DESCRIPTION OF THE PREFERRED EMBODIMENTS

Hereinafter, an embodiment of a radiological imaging system of the present invention will be described with reference to the drawings. However, the scope of the invention is not limited to the illustrated examples.

### [Configuration of an Entire Radiological Imaging System]

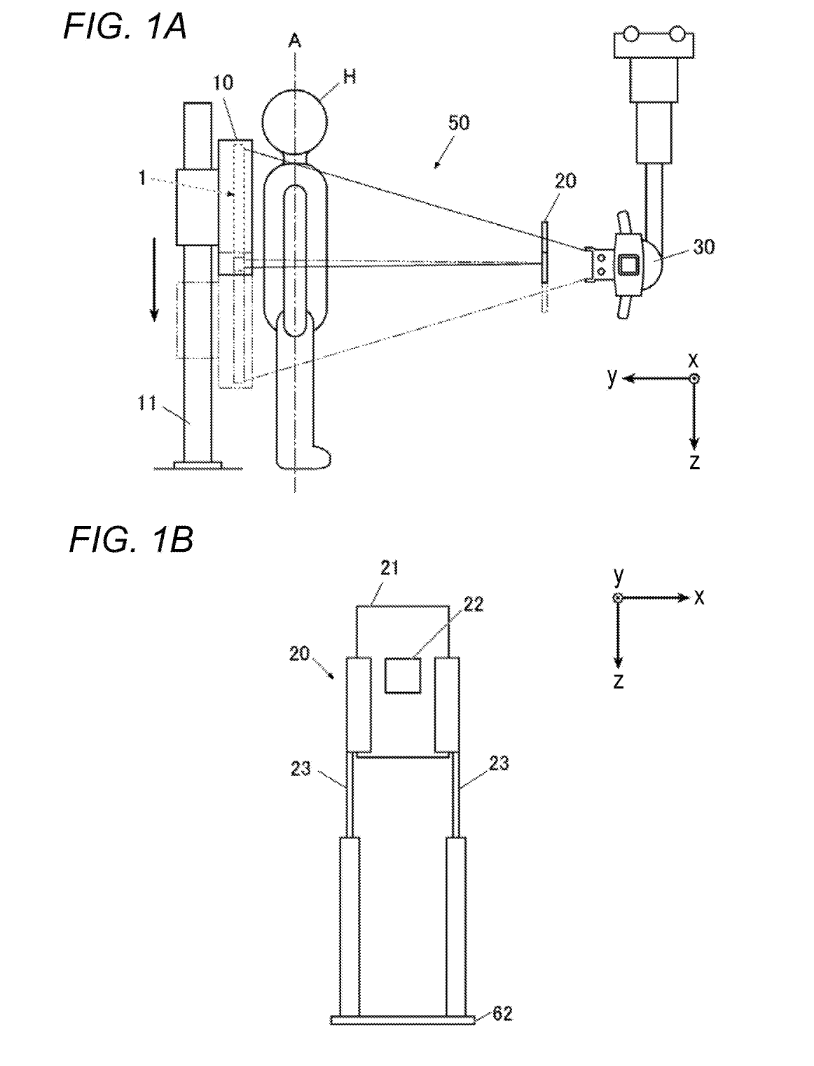

FIG. 1A is a schematic view of the configuration of an entire radiological imaging system according to this embodiment. FIG. 1B is a diagram showing an example structure of a collimator.

In the description below, an example case where imaging is performed while an object H is standing as shown in FIG. 1A, or in a so-called upright position, is described. However, imaging can be performed in the same manner while the object H is in the above described recumbent position. In the description below, an example case where a radiation source 30 is a suspension-type radiation source that is suspended from the ceiling of an imaging room is described. However, this embodiment can also be applied to a stationary radiation source or the like that is mounted on the floor surface of an imaging room.

In this embodiment, a radiological imaging system 50 includes a radiological imaging apparatus 1, a bucky 10 in which the radiological imaging apparatus 1 can be installed, a collimator 20, and the radiation source 30 that delivers radiation to the radiological imaging apparatus 1. Although not shown in the drawings, the radiological imaging system 50 also includes a control device that controls vertical movements and the like of the bucky 10 and the collimator 20, and the generator for the radiation source 30 that sets the dose of radiation to be delivered from the radiation source 30 based on a set tube voltage or tube current, an irradiation time, or the like.

In the radiological imaging apparatus 1, detection elements (not shown) are two-dimensionally arranged. When radiation is delivered, charge is generated in accordance with the dose of the radiation delivered to each of the detection elements, and image data D corresponding to the generated charge is generated. An image processing device or the like (not shown) generates a radiological image based on the image data D generated by the radiological imaging apparatus 1. In the case of long-length imaging, such radiological images are combined to generate one radiological image. The method of generating a long-length radiological image by combining two or more radiological images is a known technology disclosed in JP 2013-226243A, which has been described above, and therefore, is not described herein.

The bucky 10 is designed to vertically move along a support pillar 11 standing on the floor surface of the imaging room and stop when driven by a drive unit such as a motor (not shown), with the radiological imaging apparatus 1 being installed therein. The bucky 10 may not be designed to accommodate the so-called cassette-type radiological imaging apparatus 1, and the radiological imaging apparatus 1 may not be detachably installed in the bucky 10. Instead, the bucky 10 and the radiological imaging apparatus 1 may be integrally formed.

As shown in FIG. 1B, the collimator 20 includes a shield 21, and a rectangular aperture 22 of a predetermined size is formed in the shield 21. The size and the shape of the aperture 22, and the distance from the collimator 20 to the radiation source 30 are set so that radiation that is released from the radiation source 30 and passes through the aperture 22 is delivered to the entire region of the radiological imaging apparatus 1 in the bucky 10 or to a region slightly larger than the entire region of the radiological imaging apparatus 1.

The collimator 20 has the shield 21 supported by a support pillar 23, for example, and the shield 21 accompanied by the support pillar 23 vertically moves and stops when driven by a drive unit such as a motor (not shown). The drive unit that vertically moves the above described bucky 10 and the drive unit for the collimator 20 are made to interlock with each other, so that the collimator 20 is moved in the direction of the body axis A of the object H in synchronization with movement of the bucky 10 in the direction of the body axis A, and the collimator 20 stops in a predetermined position in the direction of the body axis A when the radiological imaging apparatus 1 stops in a predetermined position in the direction of the body axis A. When radiation is released from the radiation source 30, the radiation that has passed through the aperture 22 of the collimator 20 is delivered precisely to the radiological imaging apparatus 1.

Although the collimator 20 shown in FIG. 1B stands on the floor surface of the imaging room, the collimator 20 may be suspended from the ceiling of the imaging room, or may be integrally formed with the radiation source 30. Therefore, the structure of the collimator 20 is not limited to a type that stands on the floor surface of an imaging room.

### [Structure of a Conventional Radiation Source]

Next, the structure of a conventional radiation source 100 is described before the structure of the radiation source 30 in the radiological imaging system 50 according to this embodiment is described.

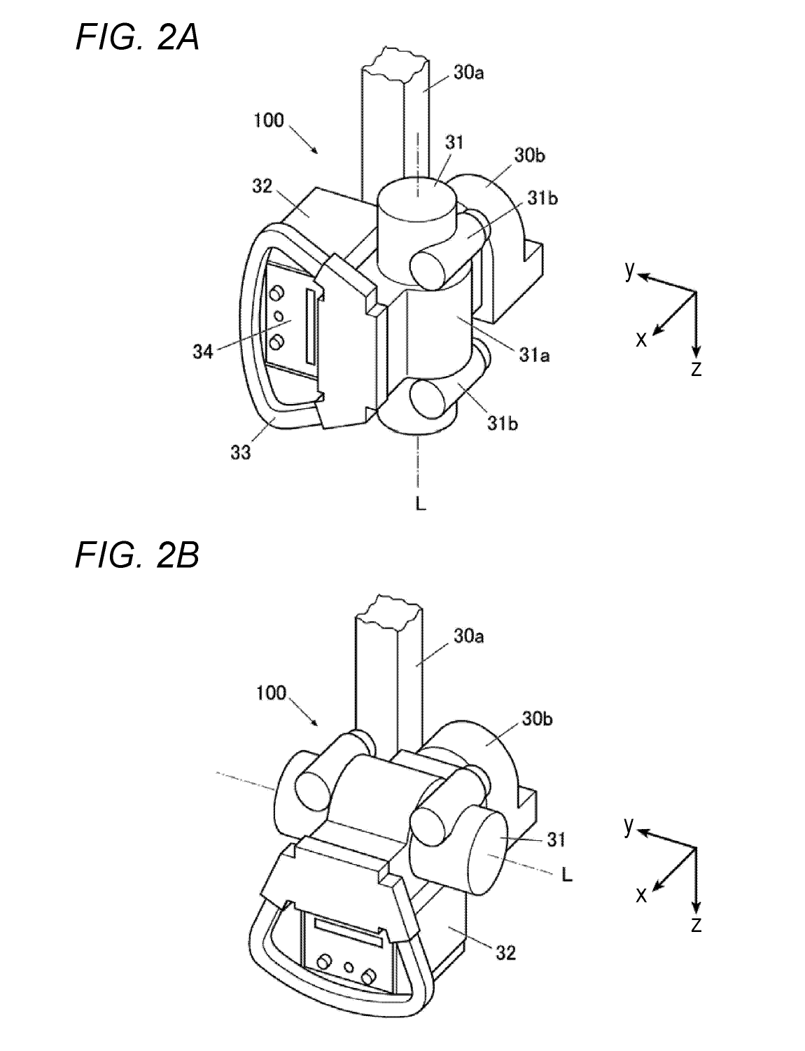

As shown in FIG. 2A, the conventional radiation source 100 includes: a substantially cylindrical frame 31 containing a rotating anode or the like described later; and a diaphragm unit 32 that sets a radiation field of the radiation to be delivered. The radiation source 100 further includes: a handle 33 to be gripped by a radiological technologist or the like to change the position or the orientation of the radiation source 100; and a panel 34 for performing various operations on the radiation source 100. The frame 31 is secured by a holder 31a, and a high-voltage cable 31b or the like for supplying a high voltage to the rotating anode or the like is further attached to the frame 31.

Rotated about a shaft 30b provided at an end (the lower end in the case shown in FIGS. 2A and 2B) of a support pillar 30a, the radiation source 100 may switch between an upright position in which radiation is released in a horizontal direction as shown in FIG. 2A, and a recumbent position in which radiation is released downward as shown in FIG. 2B. Although not shown in FIG. 2B, the object H is lying so that the body axis A extends in the y-axis direction in the drawing.

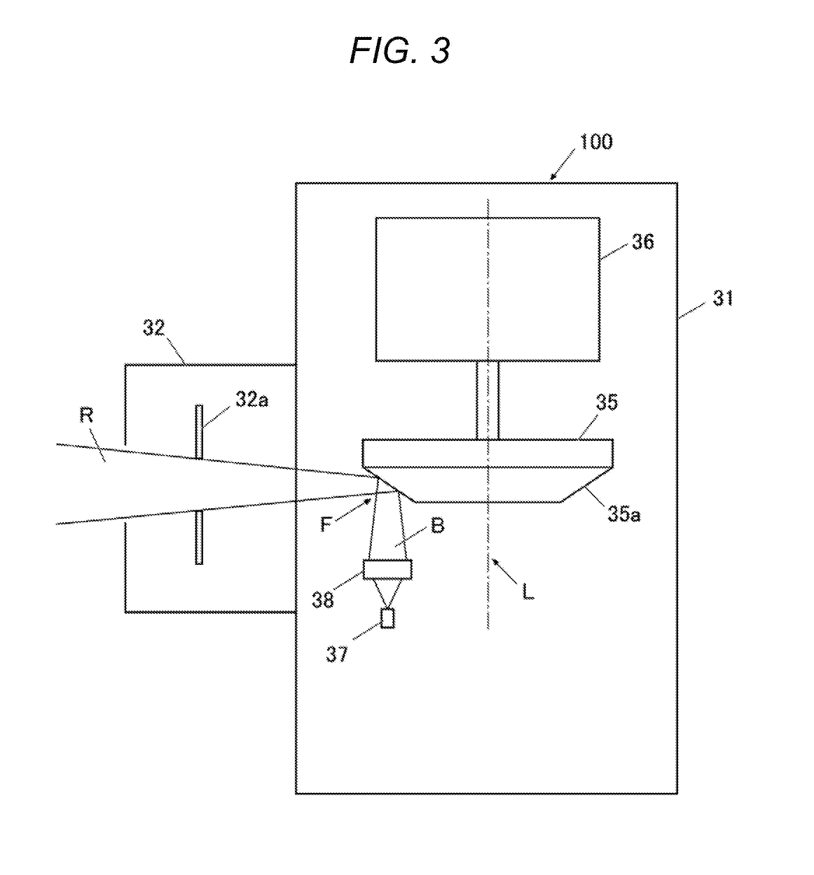

As shown in FIG. 3, a rotating anode 35 (or a tubular bulb) that has a substantially disk-like shape, is also called a rotor or the like, and is formed with tungsten (W), molybdenum (Mo), or the like is provided inside the frame 31 of the radiation source 100. The rotating anode 35 rotates about a tube axis L at a high speed when driven by a drive unit 36 including a motor or the like (not shown).

An electron gun 37 and an electron lens 38 are also provided inside the frame 31 of the radiation source 100. An electron beam B emitted from the electron gun 37 is narrowed by the electron lens 38 and is delivered to a target surface 35a formed on the rotating anode 35. As a result, radiation R such as an X-ray is generated, with the focal point F being the portion of the target surface 35a of the rotating anode 35 irradiated with the electron beam B. The radiation field of the radiation R is narrowed by a diaphragm 32a in the diaphragm unit 32, and is delivered to the object H and the radiological imaging apparatus 1 (see FIG. 1A). The conventional radiation source 100 has the above described structure.

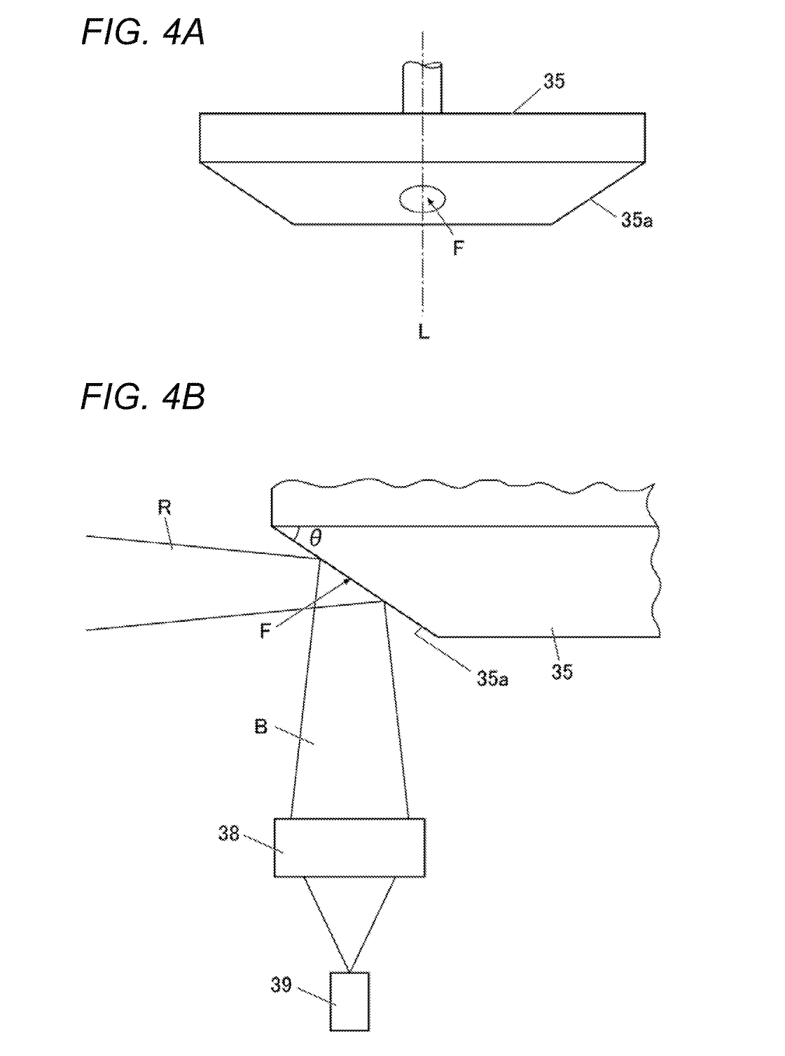

Narrowing the focal point in the radiation source 100 is now described. To make the focal point F easier to see in FIG. 3 and FIGS. 4A and 4B described later, the size of the focal point F relative to the size of the rotating anode 35 or the like in those drawings is larger than the actual size. In reality, however, the diameter of the focal point F is on the order of several hundreds of micrometers, and is extremely small.

FIG. 4A shows the focal point F of the radiation R on the rotating anode 35 of the radiation source 100, seen from the side of the object H (the left side in FIG. 3). If the width of the focal point F is too large, the captured radiological image is blurred. So as to reduce the width of the focal point F at least in the vertical direction (the direction of the tube axis L), the target surface 35a of the rotating anode 35 irradiated with the electron beam B is normally a sloping surface as shown in FIG. 4B.

Where the angle θ of the target surface 35a of the rotating anode 35 with respect to the plane of rotation (the angle θ with respect to a direction perpendicular to the tube axis L in a case where the tube axis L is the reference) becomes smaller, the width of the focal point F in the direction of the tube axis L can be made smaller. As described above, as the target surface 35a is tilted, a high-power tubular bulb with a small focal point is realized. In practice, the angle θ is set at a very small value such as 12 degrees or 14 degrees in many cases. This angle θ may be a value determined from the existing requirements in radiological imaging.

### [Heel Effect]

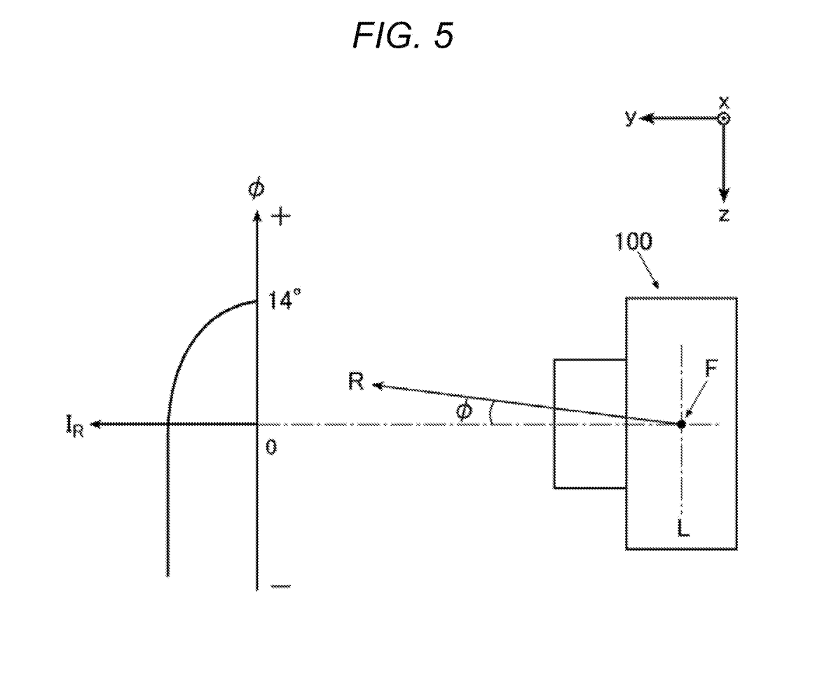

Next, the heel effect in the radiation source 100 having the above structure is described. Radiation R not narrowed by the diaphragm 32a (see FIG. 3) in the diaphragm unit 32 (the diaphragm 32a being opened at a maximum level) is released from the above described radiation source 100, and the distribution of the intensity I_{R} of the radiation R is measured. As a result, the distribution shown in FIG. 5 is obtained, for example.

Specifically, the intensity I_{R} of the radiation R released from the radiation source 100 is vertically swayed at an angle φ and is measured in a position at a predetermined distance from the radiation source 100, with the direction of the rotational plane of the rotating anode 35 (or the direction perpendicular to the tube axis L) being the reference (φ=0°). In this case, the above described angle θ of the target surface 35a of the rotating anode 35 is 14 degrees.

As shown in FIG. 4B, the target surface 35a of the rotating anode 35 is tilted so that the normal line thereof extends downward compared with the direction of the rotational plane. Therefore, in a case where the angle φ has a negative value or where the radiation R is released downward compared with the direction of the rotational plane of the rotating anode 35, the intensity I_{R} of the radiation R hardly decreases, as shown in the graph in FIG. 5. However, in a case where the angle φ has a positive value or where the radiation R is released upward compared with the direction of the rotational plane of the rotating anode 35, the intensity I_{R} of the radiation R becomes lower as the angle φ becomes larger, and the intensity I_{R} of the radiation R becomes almost zero when the angle φ is +14 degrees or greater. This is the heel effect.

As described above, the heel effect is a phenomenon that occurs when the target surface 35a of the rotating anode 35 is tilted so that the normal line thereof extends downward compared with the direction of the rotational plane. More accurately, the heel effect is a phenomenon that occurs because the target surface 35a of the rotating anode 35 is formed so that the normal line thereof is tilted in the direction toward the tube axis L rather than in the direction of the rotational plane. The heel effect is also a phenomenon that occurs in a direction parallel to the tube axis L of the rotating anode 35. It should be noted that the heel effect does not occur in a direction perpendicular to the tube axis L (a horizontal direction in FIG. 5 (or a direction perpendicular to the paper surface)).

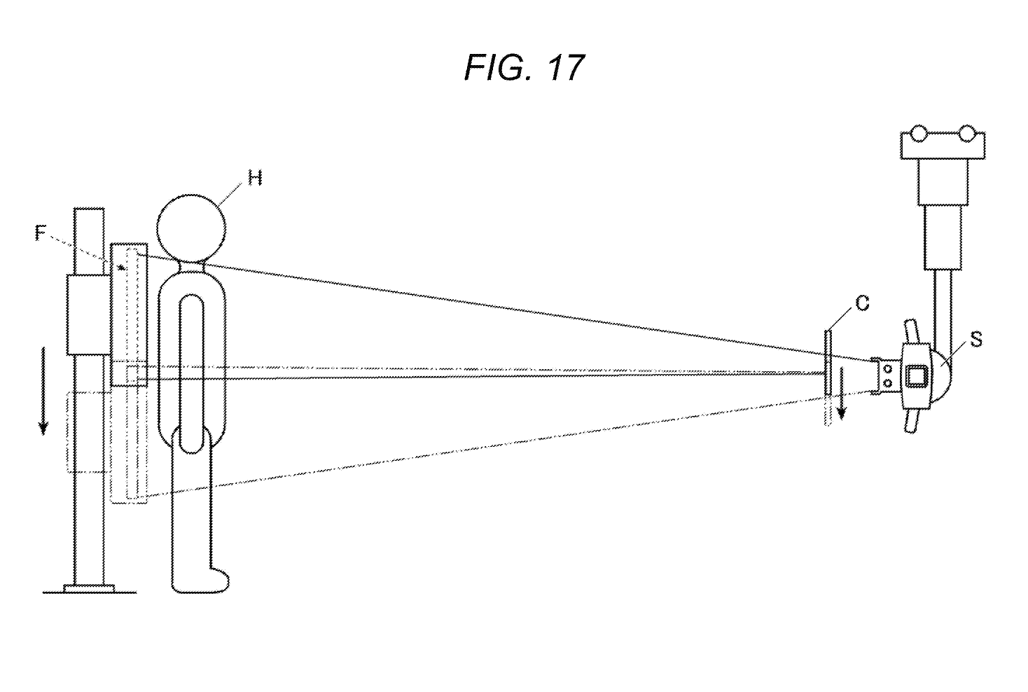

Since such a heel effect occurs, it is difficult to widen the irradiation area (or the radiation field) of the radiation R released from the radiation source 100 in the direction of the tube axis L of the rotating anode 35 (the direction of the body axis A (see FIG. 1A) of the object H positioned parallel to the tube axis L) in the conventional radiation source 100, as described above. Therefore, there are cases where the radiation source S (or the radiation source 100) must be moved far away from the radiological imaging apparatus F and the object H, and cannot be placed in a small, narrow imaging room, as shown in FIG. 17.

### [Structure of the Radiation Source According to This Embodiment]

Next, the structure of the radiation source 30 in the radiological imaging system 50 according to this embodiment is described. In the description below, the same components and the like as those in the above described conventional radiation source 100 are denoted by the same reference numerals used in the conventional radiation source 100.

In the conventional radiation source 100, the tube axis L of the rotating anode 35 is placed parallel to the body axis A of the object H, and therefore, it is difficult to widen the radiation field of the radiation R in the direction of the body axis A of the object H due to the heel effect, as described above. In view of this, the radiation source 30 (see FIG. 1A) is designed so that the tube axis L of the rotating anode 35 extends in a direction perpendicular to the body axis A (see FIG. 1A) of the object H being imaged in this embodiment. This aspect is described below in detail.

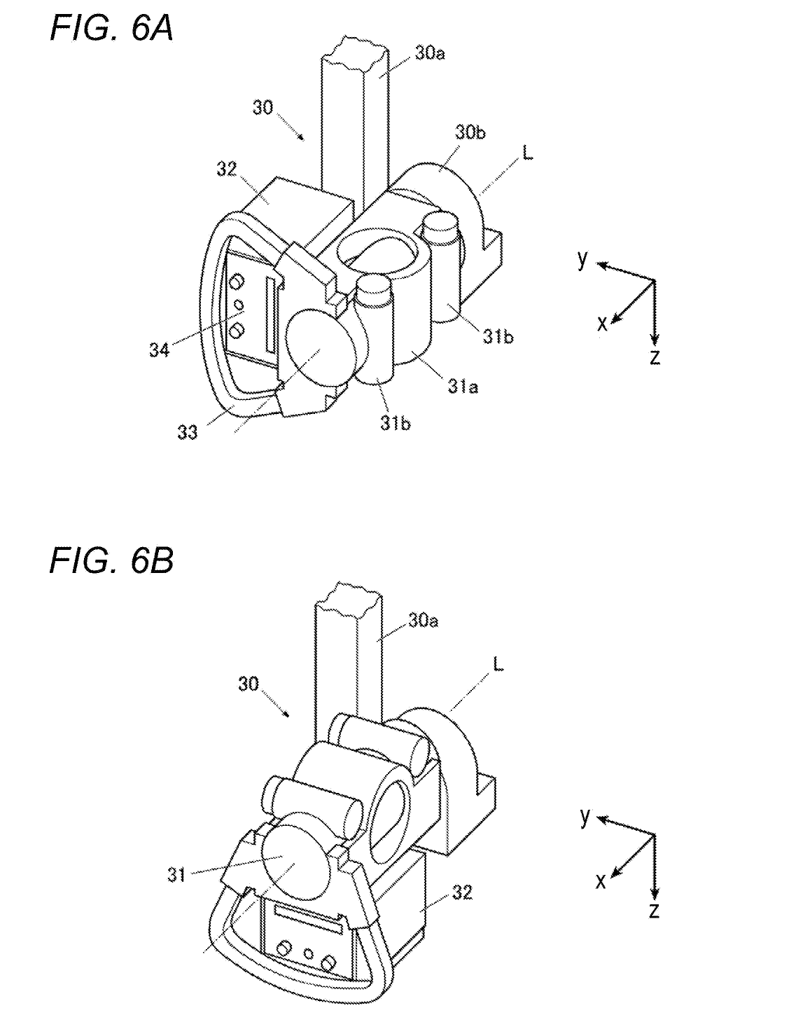

In the radiation source 30 according to this embodiment, the structure inside the frame 31 is the same as that in the conventional radiation source 100 shown in FIG. 3. As shown in FIG. 6A, like the conventional radiation source 100 shown in FIG. 2A, the radiation source 30 according to this embodiment is formed with the frame 31, the diaphragm unit 32, and the like, and further includes the handle 33, the panel 34, and the like. The frame 31 is secured by a holder 31a, and a high-voltage cable 31b or the like for supplying a high voltage to the rotating anode or the like is further attached to the frame 31.

However, the radiation source 30 according to this embodiment differs from the conventional radiation source 100 in that the cylinder-like frame 31 containing the rotating anode 35 (see FIG. 3 and others) and the like is placed so that the tube axis L of the rotating anode 35 extends in a direction perpendicular to the body axis A of the object H.

FIG. 6A and others show a case where the holder 31a is designed so that the position of attachment of the frame 31 to the holder 31a can be changed. Specifically, in FIG. 6A and others, the holder 31a is designed so that the position of attachment of the frame 31 to the holder 31a is changed, and accordingly, the radiation source can be used as the radiation source 30 according to this embodiment shown in FIG. 6A and others, or as the conventional radiation source 30 shown in FIG. 2A and others. However, the holder 31a may not be designed so that the position of attachment of the frame 31 can be changed, but may be designed especially for the radiation source 30 according to this embodiment.

Like the above described conventional radiation source 100 (see FIG. 2B), the radiation source 30 according to this embodiment can be designed to switch between an upright position in which radiation is released in a horizontal direction as shown in FIG. 6A and a recumbent position in which radiation is released downward as shown in FIG. 6B, when rotated about a first shaft 30b provided at an end (the lower end in the case shown in FIGS. 6A and 6B) of a support pillar 30a, for example.

Although not shown in FIG. 6B, the object H is also lying in this case so that the body axis A extends in the y-axis direction in the drawing, as in the case of the conventional radiation source 100 described above with reference to FIG. 2B. Accordingly, in the recumbent position shown in FIG. 6B, the radiation source 30 is also placed so that the tube axis L of the rotating anode 35 extends in a direction perpendicular to the body axis A of the object H being imaged.

### [Functions]

Next, the functions of the radiological imaging system 50 according to this embodiment are described. In this embodiment, the radiation source 30 is placed so that the tube axis L of the rotating anode 35 extends in a direction perpendicular to the body axis A of the object H as described above. Since the above described heel effect occurs in the direction of the tube axis L, any heel effect does not occur in the direction of the body axis A (see FIG. 1A, for example) of the object H perpendicular to the tube axis L.

That is, in the radiation source 30 according to this embodiment, any heel effect does not occur at least in the direction of the body axis A of the object H. Therefore, a heel effect occurs in the direction of the body axis A of the object H, and the radiation source 30 is not prevented from widening the radiation field in the direction of the body axis A of the object H, unlike the above described conventional radiation source 100.

Accordingly, in the radiation source 30 according to this embodiment, there is no need to increase the distance between the radiation source S and the object H or the like so as to widen the radiation field in the direction of the body axis A of the object H as in the conventional radiation source S shown in FIG. 17 (or the above described radiation source 100), and the distance between the radiation source 30 and the object H or the radiological imaging apparatus 1 or the like can be shortened while the radiation field is sufficiently widened in the direction of the body axis A of the object H as shown in FIG. 1A.

### [Effects]

As described above, in the radiological imaging system 50 according to this embodiment, the radiation source 30 is placed so that the tube axis L of the rotating anode 35 extends in a direction perpendicular to the body axis A of the object H. Accordingly, any heel effect can be prevented from occurring at least in the direction of the body axis X of the object H, and the field of radiation to be released from the radiation source 30 can be sufficiently and precisely widened in the direction of the body axis A of the object H.

Thus, the distance between the radiation source 30 and the object H or the radiological imaging apparatus 1 or the like can be further shortened, and the collimator-type radiation source 30 can be placed in a small, narrow imaging room. In this manner, radiological imaging for long-length imaging or the like can be accurately performed.

### [Other Modes of Long-Length Imaging]

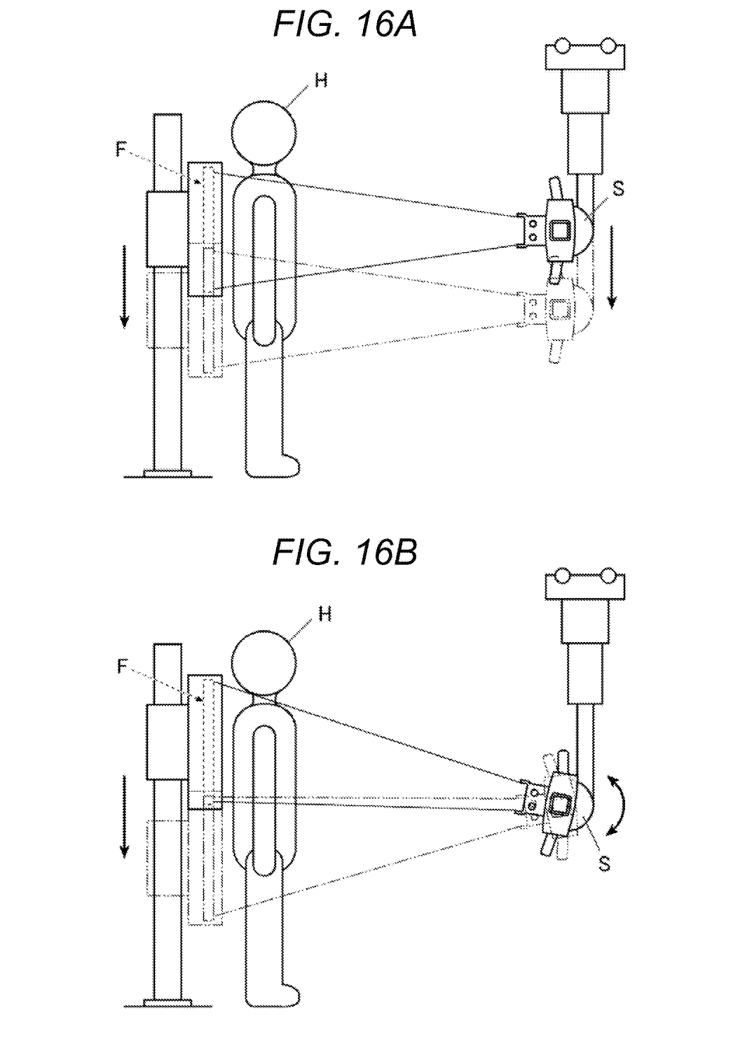

In the conventional radiation source 100, the field of radiation to be released from the radiation source 100 cannot be sufficiently widened in the direction of the body axis A of the object H due to the heel effect as described above. Therefore, the radiation source 100 is moved in the vertical direction (see FIG. 16A), or the head of the radiation source 100 is swayed (see FIG. 16B), so that radiation is released from the radiation source 100 twice or more to perform long-length imaging.

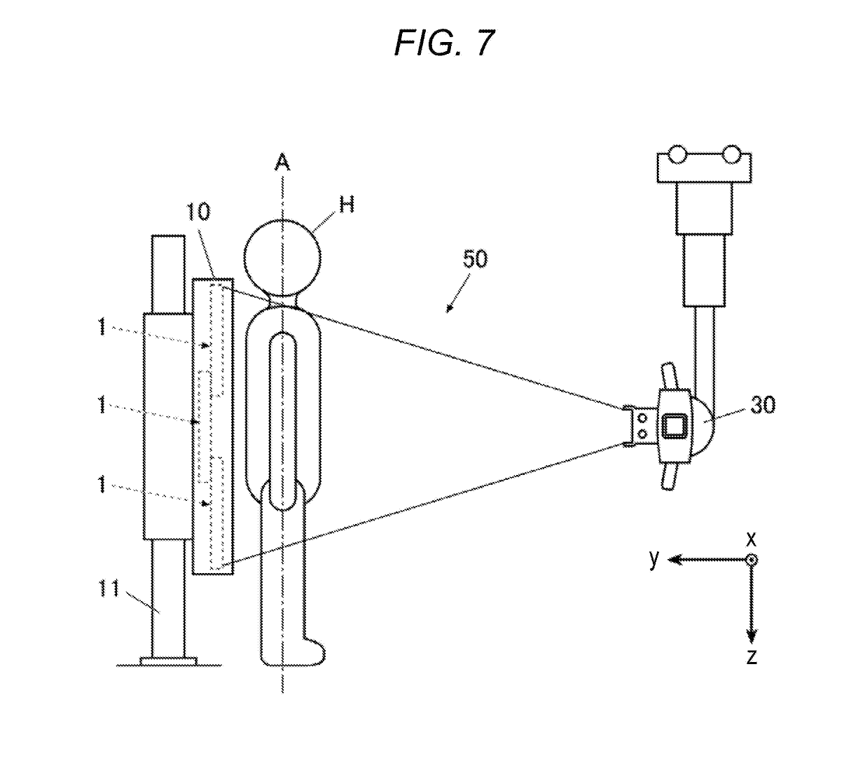

In this embodiment, on the other hand, the field of radiation to be released from the radiation source 30 can be sufficiently widened in the direction of the body axis A of the object H as described above. Accordingly, radiological imaging apparatuses 1 can be arranged behind the object H in the direction of the body axis A of the object H as shown in FIG. 7, for example, and long-length imaging can be performed by delivering radiation from the radiation source 30 to the radiological imaging apparatuses 1 via the object H only once.

With this configuration, radiation needs to be released from the radiation source 30 only once in long-length imaging. In conventional long-length imaging where radiation is released from the radiation source twice or more, the object H moves before the next radiation is released, or there is the problem of body movement. In the above described case, on the other hand, radiation is released only once, and the problem of body movement does not arise.

In the conventional long-length imaging, a patient as the object H is required not to move his/her body until the multiple release of radiation is ended. However, where radiation is released only once in long-length imaging as described above, the patient needs to stay still only during the “one-time shooting”. Accordingly, the stress on the patient can be reduced.

Furthermore, in the configuration shown in FIG. 7, there is no need to prepare the collimator 20 (see FIGS. 1A and 1B), and the radiological imaging apparatus 1 and the collimator 20 do not need to move and stop in synchronization with each other as in the case illustrated in FIG. 1A and others. Accordingly, the control structure in the long-length imaging using the radiological imaging system 50 becomes advantageously simpler.

The structure of the imaging stand for long-length imaging in a case where radiological imaging apparatuses 1 are arranged in the direction of the body axis A of the object H, and radiation is delivered from the radiation source 30 to the radiological imaging apparatuses 1 via the object H only once, or “one-time shooting” is performed, will be described later in detail.

### [Use of the Radiological Imaging System in Imaging Other Than Long-Length Imaging]

In the above description, long-length imaging is performed with the radiological imaging system 50 according to this embodiment. However, the radiological imaging system 50 and the radiation source 30 according to this embodiment can be used not only in long-length imaging but also in general imaging for taking a single radiological image and other various kinds of radiological imaging. This embodiment is applied in cases where the radiological imaging system 50 is used in imaging other than long-length imaging.

With a conventional radiation source, radiological imaging is performed by delivering radiation in a radiation field that is narrow at least in the direction of the body axis A of the object H due to a heel effect, as described above. In a case where general imaging or the like is performed with the radiological imaging system 50 according to this embodiment, on the other hand, radiological imaging can be performed by delivering radiation from the radiation source 30 while the radiation field can be sufficiently and precisely widened in the direction of the body axis A of the object H as described above.

### [Switching Between a Conventional Structure and a Structure According to This Embodiment]

The radiation source 30 can be switched between a position in which the tube axis L of the rotating anode 35 extends in a direction parallel to the body axis A of the object H as in a conventional case, and a position in which the tube axis L of the rotating anode 35 extends in a direction perpendicular to the body axis A of the object H as in this embodiment.

### [Switching Method 1]

In this case, the holder 31a for securing the frame 31 containing the rotating anode 35 and the like can secure the frame 31 so that the tube axis L of the rotating anode 35 extends in a direction perpendicular to the body axis A of the object H (see FIGS. 6A and 6B), and can also secure the frame 31 so that the tube axis L of the rotating anode 35 extends in a direction parallel to the body axis A of the object H (see FIGS. 2A and 2B), for example.

In such a structure, the position of the frame 31 with respect to the holder 31a can be changed, and the direction of the tube axis L of the rotating anode 35 of the radiation source 30 can be changed as described above. In this case, the high-voltage cable 31b needs to be reattached, for example.

### [Switching Method 2]

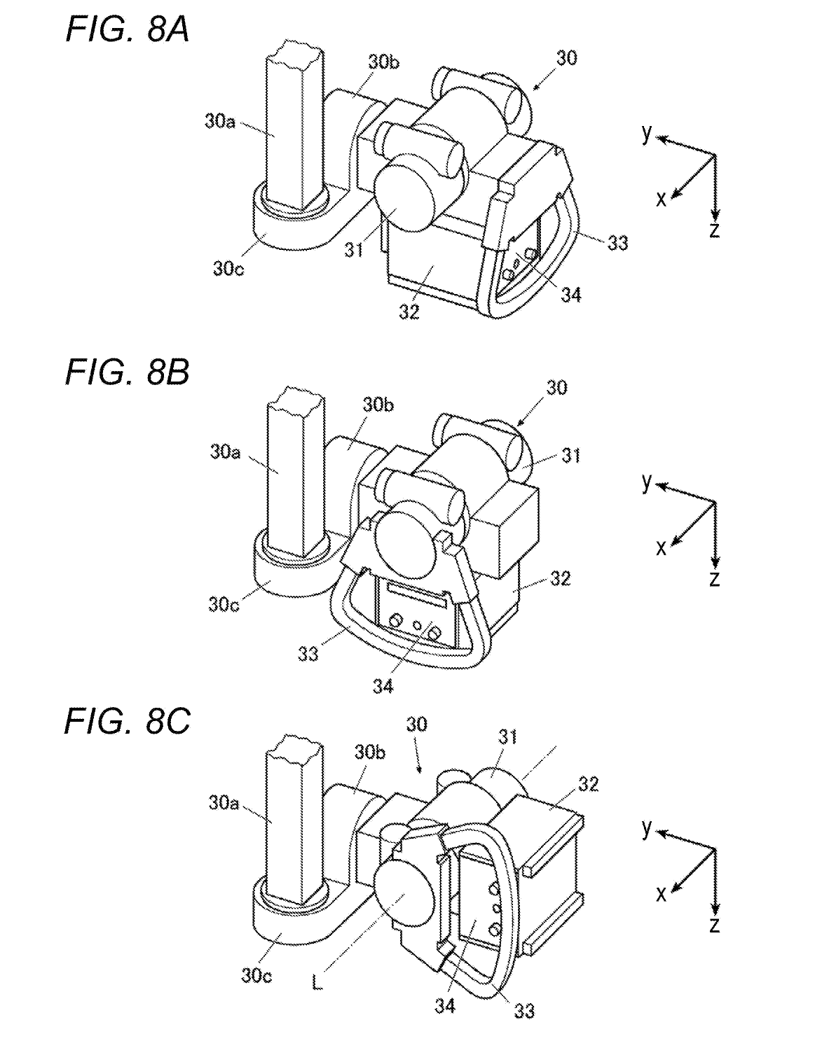

By another method of changing the direction of the tube axis L of the rotating anode 35, the radiation source 30 in the position of the conventional radiation source shown in FIG. 2A is rotated 90 degrees downward about the first shaft 30b, to be moved to the conventional recumbent position shown in FIG. 2B. After the radiation source 30 in that state is horizontally rotated 90 degrees about a second shaft 30c as shown in FIG. 8A, the handle 33 and the panel 34 are removed and are reattached to a side of the radiation source 30 as shown in FIG. 8B.

The radiation source 30 in that state is then rotated 90 degrees to change the direction of release of radiation from a downward direction to a horizontal direction, as shown in FIG. 8C. In this manner, the tube axis L of the rotating anode 35 can be made to extend in a direction perpendicular to the body axis A of the object H as in this embodiment. Alternatively, the orientation of the radiation source 30 may be changed in reverse manner of the above described manner, so that the radiation source 30 can be returned to the conventional state where the tube axis L of the rotating anode 35 extends in a direction parallel to the body axis A of the object H as shown in FIGS. 2A and 2B.

In the above described structure, the direction of the tube axis L of the rotating anode 35 of the radiation source 30 can be changed in the above manner. In this case, the direction of release of radiation is the opposite of the direction of release of radiation in the case illustrated in FIG. 2A, and the handle 33 and the panel 34 need to be reattached (see FIG. 8B), for example. Furthermore, there is the need to prepare a mechanism for changing the direction of release of radiation from a downward direction to a horizontal direction by rotating the radiation source 30 90 degrees, as shown in FIG. 8C. In a case where the radiation source 30 is used only in a recumbent position as shown in FIG. 8B, there is no need to prepare this new mechanism.

### [Switching Method 3]

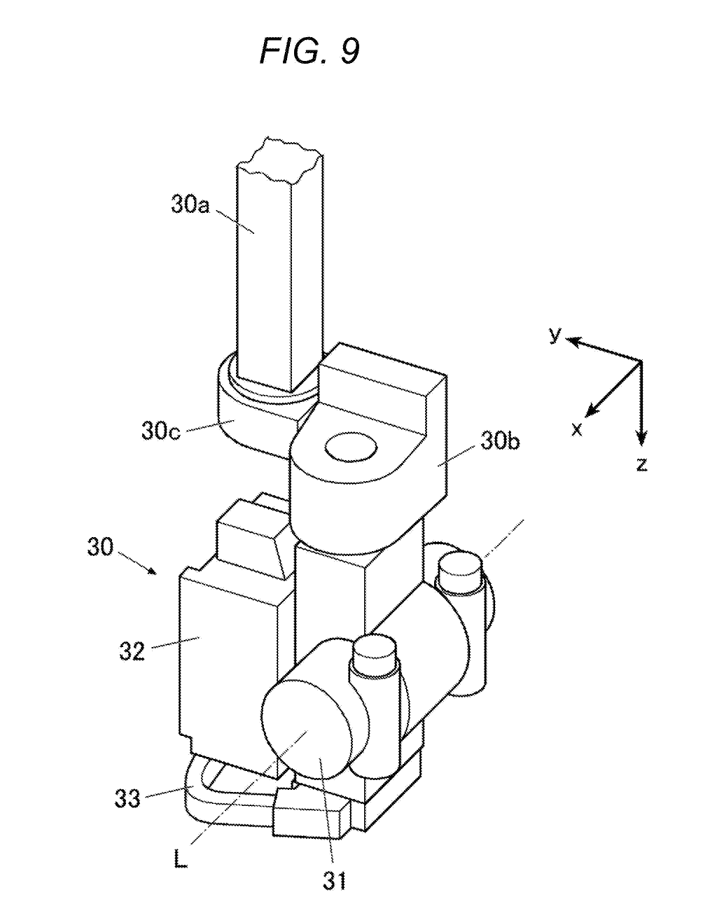

By yet another method of changing the direction of the tube axis L of the rotating anode 35, the first shaft 30b is rotated 90 degrees about the second shaft 30c in the conventional radiation source state shown in FIG. 2A, for example, so that the tube axis L of the rotating anode 35 can be made to extend in a direction perpendicular to the body axis A of the object H as in this embodiment, as shown in FIG. 9. Although not shown in any drawing, the handle 33 and the panel 34 also need to be reattached in this case.

In this case, the conventional state shown in FIG. 2A can also be recovered from the state of this embodiment shown in FIG. 9 when the first shaft 30b is rotated 90 degrees in reverse direction about the second shaft 30c. In the above described structure, the direction of the tube axis L of the rotating anode 35 of the radiation source 30 can be changed. In this case, there is the need to prepare a mechanism for rotating the first shaft 30b about the second shaft 30c.

In the above manner, the radiation source 30 can be switched between a position in which the tube axis L of the rotating anode 35 extends in a direction parallel to the body axis A of the object H as in a conventional case, and a position in which the tube axis L of the rotating anode 35 extends in a direction perpendicular to the body axis A of the object H as in this embodiment. It is also possible to design a structure so as to switch positions by a method other than any of the above described methods.

### [Image Data Correction Accompanied by a Heel Effect]

In the radiation source 30 according to this embodiment, any heel effect (see FIG. 5) does not occur at least in the direction of the body axis A of the object H, but a heel effect occurs in a direction perpendicular to the body axis A of the object H or the direction of the tube axis L (see FIGS. 6A and 6B) of the rotating anode 35 of the radiation source 30.

Therefore, in a case where the radiological imaging system 50 according to this embodiment captures an image of the object H by releasing radiation from the radiation source 30, the right side or the left side of the object H is dark in the radiological image. That is, the intensity I_{R} (see FIG. 5) of the radiation that reaches the right side or the left side of the radiological imaging apparatus 1 becomes lower due to a heel effect. This phenomenon occurs in both cases where imaging is performed in an upright position and where imaging is performed in a recumbent position.

Therefore, image data D generated in the radiological imaging apparatus 1 can be corrected at least in a direction perpendicular to the body axis A of the object H based on the distribution of the intensity I_{R} of radiation released from the radiation source 30 in the direction perpendicular to the body axis A of the object H. This image correction may be performed by the radiological imaging apparatus 1, or may be performed by an image processing device (not shown) that receives the image data D transferred from the radiological imaging apparatus 1.

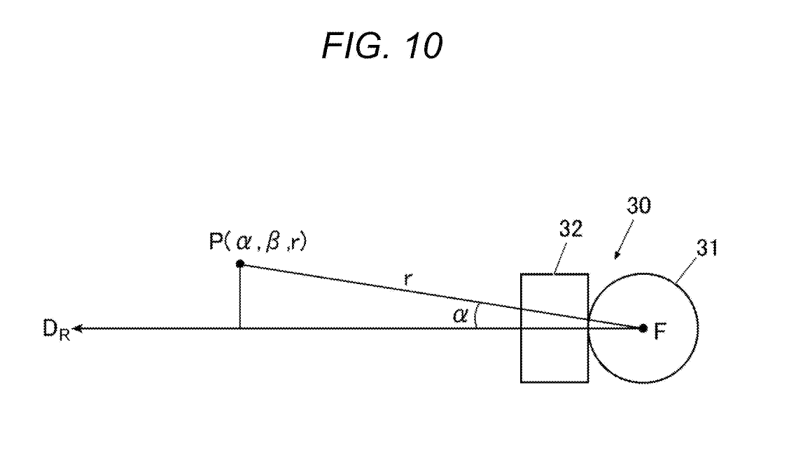

Specifically, a correction value v can be calculated beforehand for each position P with respect to the radiation source 30. In this case, a position P with respect to the radiation source 30 can be represented by P (α, β, r), where a represents the angle in the vertical direction with respect to the direction D_{R} of release of radiation from the radiation source 30, β represents the angle (not shown) in the horizontal direction with respect to the direction D_{R} of release of radiation, and r represents the distance from the focal point F of the radiation source 30, as shown in FIG. 10, for example.

When radiation of a predetermined intensity I_{R}O is released from the radiation source 30, the intensity I_{R} (α, β, r) of the radiation is measured at each position P (α, β, r), and I_{R}O/I_{R} (α, β, r) or a value obtained by multiplying I_{R}O/I_{R} (α, β, r) by a constant can be used as the correction value v.

In this case, a heel effect appears on the right or left side when the object H is seen from the radiation source 30, or in a direction perpendicular to the body axis A of the object H, as described above in this embodiment. Where the intensity I_{R} of the radiation is measured at each position P in the above manner, the intensity I_{R} of the radiation that has reached a right- or left-side position P is smaller. Therefore, the correction value v is set so as to be greater where the intensity I_{R} of the arriving radiation becomes lower due to a heel effect.

The radiological imaging apparatus 1 or the image processing device calculates the correction value v beforehand for each position P with respect to the radiation source 30 based on the distribution of the intensity I_{R} of radiation released from the radiation source 30 in a direction perpendicular to the body axis A of the object H in the above described manner. Alternatively, the correction value v calculated for each position P is obtained in advance.

After the image data D is generated by the radiological imaging apparatus 1 as a result of radiological imaging, the position P (α, β, r) of each detection element of the radiological imaging apparatus 1 with respect to the radiation source 30 is determined, and the image data D is multiplied by the correction value v for the position P. In this manner, the image data D generated by the radiological imaging apparatus 1 is corrected in the direction perpendicular to the body axis A of the object H.

However, it is not necessarily easy to measure the intensity I_{R} of radiation released from the radiation source 30 at each position P (α, β, r) with respect to the radiation source 30, and determine the distribution of the intensity I_{R} of the radiation released from the radiation source 30. In view of this, the correction value v for each position P with respect to the radiation source 30 can be calculated in the following manner.

Specifically, the radiological imaging apparatus 1 is placed in front of the radiation source 30 in the release direction D_{R}, and is moved as close to the radiation source 30 as possible, though not illustrated in any of the drawings. Radiation is then delivered from the radiation source 30 to the radiological imaging apparatus 1, and the image data D of the respective detection elements is generated. The image data D in this case is the values corrected with gain calibration data of the respective detection elements.

The radiological imaging apparatus 1 in that position is then moved in four directions (upward, downward, rightward, and leftward in the case of an upright position) perpendicular to the release direction D_{R} of radiation source 30, and generates the image data D at the respective positions. Instead of the four directions, it is possible to move the radiological imaging apparatus 1 in eight directions including the intermediate directions (specifically, in the case of an upright position, the radiological imaging apparatus 1 may be moved not only upward, downward, rightward, and leftward, but also to the upper right, the upper left, the lower right, and the lower left).

The distribution of the image data D generated at the respective positions by the radiological imaging apparatus 1 moved in the above manner is the distribution of the intensity I_{R} of radiation released from the radiation source 30 in a plane perpendicular to the release direction D_{R} of the radiation source 30 at the position closest to the radiation source 30.

The same operation as above is also performed by placing the radiological imaging apparatus 1 in front of the radiation source 30 in the release direction D_{R} and moving the radiological imaging apparatus 1 at the longest possible distance from the radiation source 30. In the operation, the radiological imaging apparatus 1 is further moved in four or eight directions perpendicular to the release direction D_{R} of the radiation source 30. In this case, the distribution of the image data D generated at the respective positions by the radiological imaging apparatus 1 is the distribution of the intensity I_{R} of radiation released from the radiation source 30 in a plane perpendicular to the release direction D_{R} of the radiation source 30 at the position farthest from the radiation source 30.

The distribution of the intensity I_{R} of radiation at distances between the shortest distance and the longest distance can be determined through interpolation with the distribution of the intensity I_{R} of radiation at the shortest distance and the distribution of the intensity I_{R} of radiation at the longest distance. In this manner, the intensity I_{R} of radiation released from the radiation source 30 at each position P (α, β, r) with respect to the radiation source 30 can be determined with the radiological imaging apparatus 1.

The correction value v for each position P with respect to the radiation source 30 is calculated beforehand in the above described manner from the obtained distribution of the intensity I_{R} of radiation, and the generated image data D can be accurately corrected with the correction value v in the image processing after the imaging.

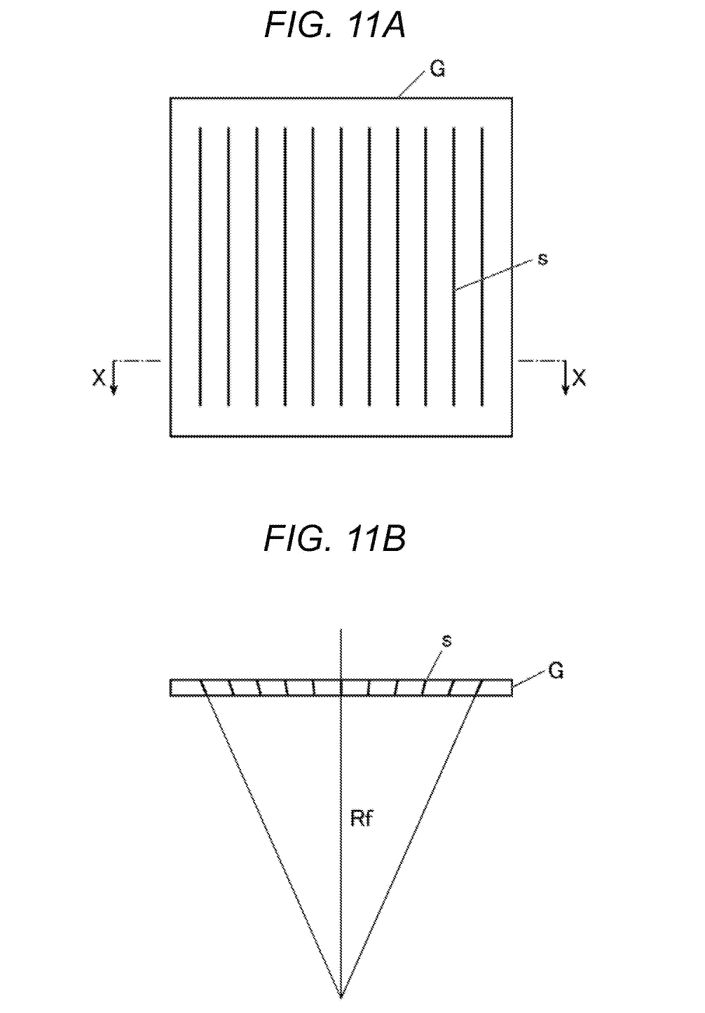

In many cases, a grid G having slits S is attached to the radiological imaging apparatus 1 during imaging as shown in FIG. 11A, for example. As shown in the cross-sectional view in FIG. 11B, a focal length Rf is set for the grid G, and the respective slits s are tilted in accordance with the focal length Rf.

Accordingly, if the distance between the grid G attached to the radiological imaging apparatus 1 and the radiation source 30 is equal to the focal length Rf of the grid G, radiation released from the radiation source 30 certainly passes through the grid G and enters the radiological imaging apparatus 1. However, if the distance between the grid G and the radiation source 30 is longer or shorter than the focal length Rf of the grid G, radiation released from the radiation source 30 is blocked by the grid G, particularly at the periphery of the grid G. As a result, radiation that enters the radiological imaging apparatus 1 at the periphery of the grid G becomes weaker.

In view of the above, when the distribution of the intensity I_{R} of radiation is determined by placing the radiological imaging apparatus 1 at the shortest distance and the longest distance from the radiation source 30 as described above, the distribution of the intensity I_{R} of radiation is determined after the grid G is attached to the radiological imaging apparatus 1. In this manner, as well as the distribution of the intensity I_{R} of radiation due to the above described heel effect, the distribution of the intensity I_{R} of radiation at respective distances can be obtained in advance, inclusive of the variation caused in the radiation that enters the radiological imaging apparatus 1 by the variation in the distance between the grid G and the radiation source 30.

A correction value v* for each position P with respect to the radiation source 30 in the case where the grid G is attached to the radiological imaging apparatus 1 can be calculated beforehand based on the above distributions of the intensity I_{R} of radiation. If the generated image data D is corrected with the correction value v*, image correction can be performed, with not only the heel effect but also the influence of the variation in the radiation intensity I_{R} due to the grid G being taken into account.

Since the above focal length Rf varies among respective grids G, the correction value v* is preferably calculated beforehand for each grid G (or for each focal length Rf).

It is also possible to obtain a correction value vg affected only by the variation in the radiation intensity I_{R} due to the grid G, instead of the correction value v* for correction that takes into account the influence of a heel effect and the influence of the variation in the radiation intensity I_{R} due to the grid G. In this case, the relationship between the correction value v affected only by a heel effect without a grid, and the correction value v* is expressed as v*=v×vg.

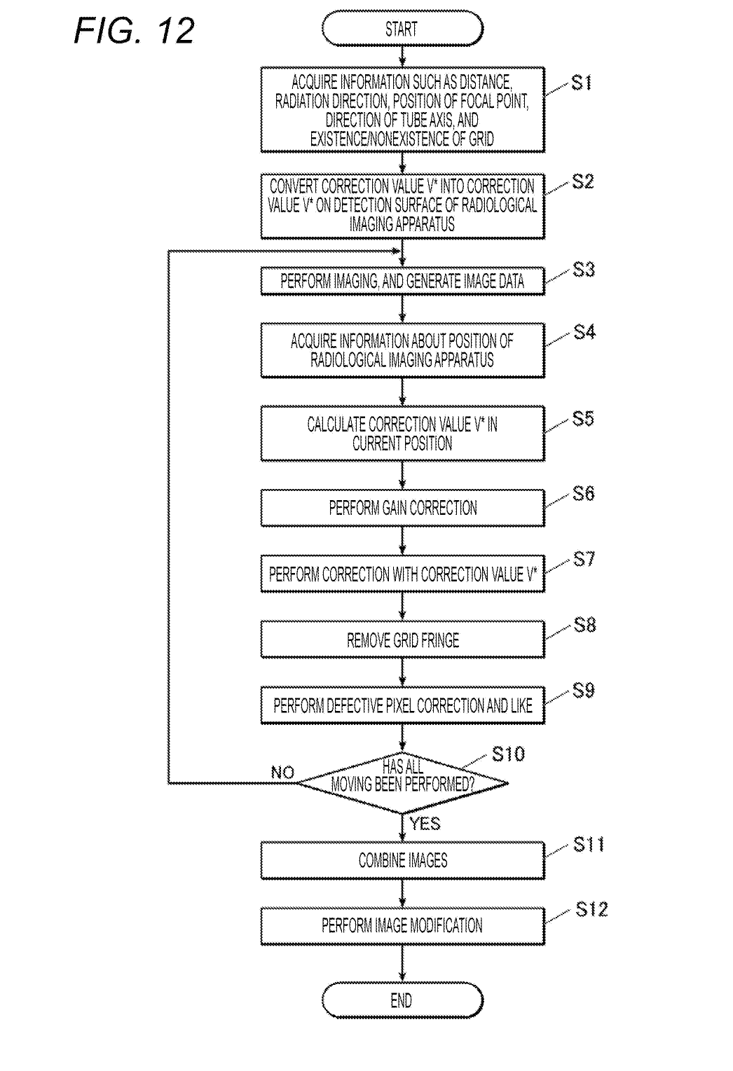

Referring now to the flowchart shown in FIG. 12, a specific example of an image generation process in the case of long-length imaging is described. In the description below, image processing is performed during each imaging process in the long-length imaging. However, image processing can be performed after a series of imaging processes is completed in the long-length imaging.

Prior to imaging, the image processing device or the radiological imaging apparatus 1 first obtains information such as the distance between the focal point F of the radiation source 30 and the radiological imaging apparatus 1, the direction of release of radiation, the spatial position of the focal point F, the direction of the tube axis L, and the existence/nonexistence of attachment of a grid G (step S1). The correction value v* in the above described case where there is a grid for each detection element is converted into a correction value v* on the detection surface of the radiological imaging apparatus 1 (or the surface on which the detection elements are two-dimensionally arranged) based on the above information such as the direction of release of radiation, the position of the focal point F, and the direction of the tube axis L (step S2).

In the description below, an example case where a grid G is attached to the radiological imaging apparatus 1 is described. However, in a case where any grid G is not attached to the radiological imaging apparatus 1, the correction value v for no-grid cases is used as the correction value, and the process related to the grid G shown in FIG. 12 is skipped.

The first imaging (or release of radiation) in the long-length imaging is performed, and image data is generated by the radiological imaging apparatus 1 (step S3). The radiological imaging apparatus 1 or the image processing device that has received the image data transferred from the radiological imaging apparatus 1 then acquires the information about the position of the radiological imaging apparatus 1 during the first imaging (step S4), and calculates the correction value v* in the current position from among correction values v* converted in the above described manner (step S5).

Gain correction (or correction based on the above mentioned gain calibration data) is then performed on the image data of each pixel (or each detection element) of the radiological imaging apparatus 1 (step S6). Each piece of the image data subjected to the gain correction is then multiplied by the correction value v* calculated in the above manner, so that the correction with the correction value v* is performed (step S7).

In the case where any grid G is not attached to the radiological imaging apparatus 1, correction is performed with a correction value v, instead of the correction value v*. In the case where the grid G is attached to the radiological imaging apparatus 1 as described above, if the correction value is in the form of a correction value v and a correction value vg, instead of the correction value v*, each piece of the image data subjected to the gain correction is multiplied by the correction value v and the correction value vg, so that correction is performed.

Since gradation due to the grid fringe is formed in the image data as is well known in the case where the grid G is attached to the radiological imaging apparatus 1, a process for removing the gradation due to the grid fringe from the image data is performed (step S8). In the case where any grid G is not attached to the radiological imaging apparatus 1, the process in step S8 is skipped.

Necessary processes such as defective pixel correction are performed (step S9), and a radiological image is generated. If not all the moving of the radiological imaging apparatus 1 has not been performed yet, and more imaging is performed in at least one different position (step S10: No), the radiological imaging apparatus 1 is moved to the next position, and the above described processes in steps S3 through S9 are repeated.

If all the moving of the radiological imaging apparatus 1 is performed (step S10: Yes), a series of imaging processes in the long-length imaging comes to an end. The radiological images generated in the respective positions of the radiological imaging apparatus 1 are then combined (step S11). The image modification necessary for making the combining portions less noticeable or the like is performed (step S12), and a single long-length radiological image is generated. The image processing then comes to an end.

### [Structure of an Imaging Stand for Long-Length Imaging by One-Time Shooting]

Next, a specific example structure of an imaging stand that is used in a case where radiological imaging apparatuses 1 are arranged in the direction of the body axis A of the object H, and long-length imaging is performed by “one-time shooting” is described.

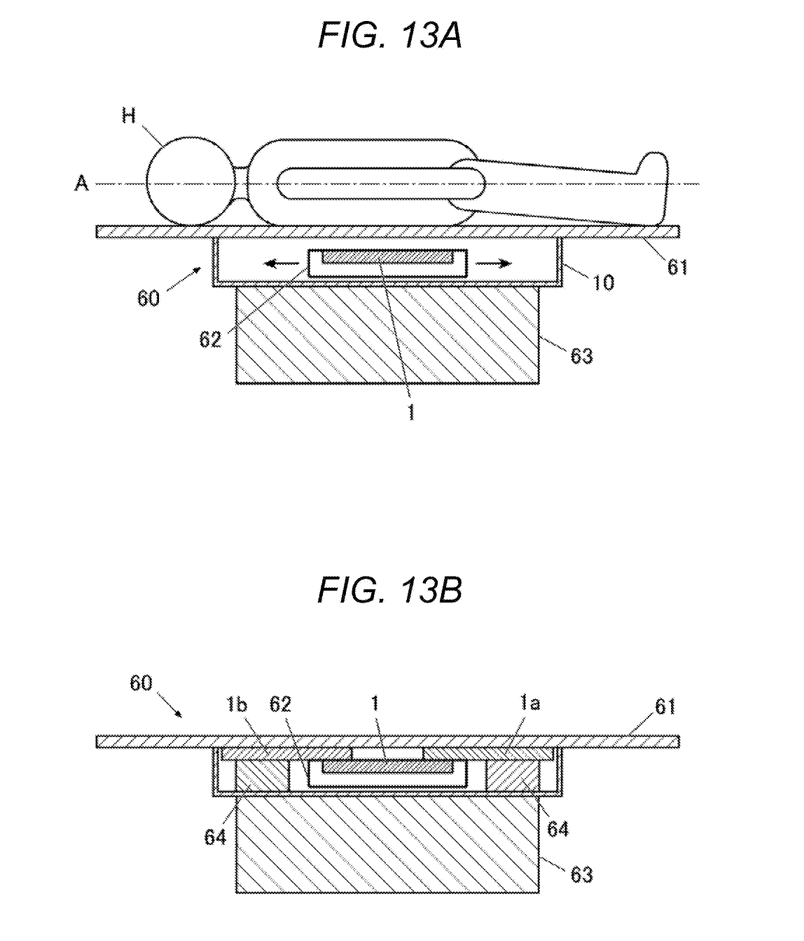

FIGS. 13A and 13B are cross-sectional views of an example structure of an imaging stand for recumbent imaging. FIG. 13A illustrates a case where a single radiological imaging apparatus is installed in the imaging stand. FIG. 13B illustrates a case where radiological imaging apparatuses are installed in the imaging stand. In this example structure, the imaging stand 60 includes a top panel 61 on which the object H is lying as shown in FIG. 13A.

A bucky 10 is placed under the top panel 61, and a radiological imaging apparatus 1 housed in a detector holder 62, for example, is installed in the bucky 10. As shown in FIG. 13A, for example, the detector holder 62 holding the radiological imaging apparatus 1 can be moved inside the bucky 10. Accordingly, the position of the radiological imaging apparatus 1 inside the bucky 10 can be changed. A mount 63 including an elevating device (not shown) is provided under the bucky 10, and the top panel 61 and the bucky 10 are moved up and down by the elevating device, so that the positions of the top panel 61 and the bucky 10 can be adjusted in the vertical direction.

As shown in FIG. 13B, radiological imaging apparatuses 1, 1a, . . . can also be installed in the bucky 10 in this example structure of the imaging stand 60. Specifically, the detector holder 62 is secured to the center position of the bucky 10, so that the position of the radiological imaging apparatus 1 is fixed. The radiological imaging apparatuses 1a and 1b are then placed in positions adjacent to the radiological imaging apparatus 1 in the direction of the body axis A of the object H, and each of the radiological imaging apparatuses 1a and 1b is secured by a position securing member 64.

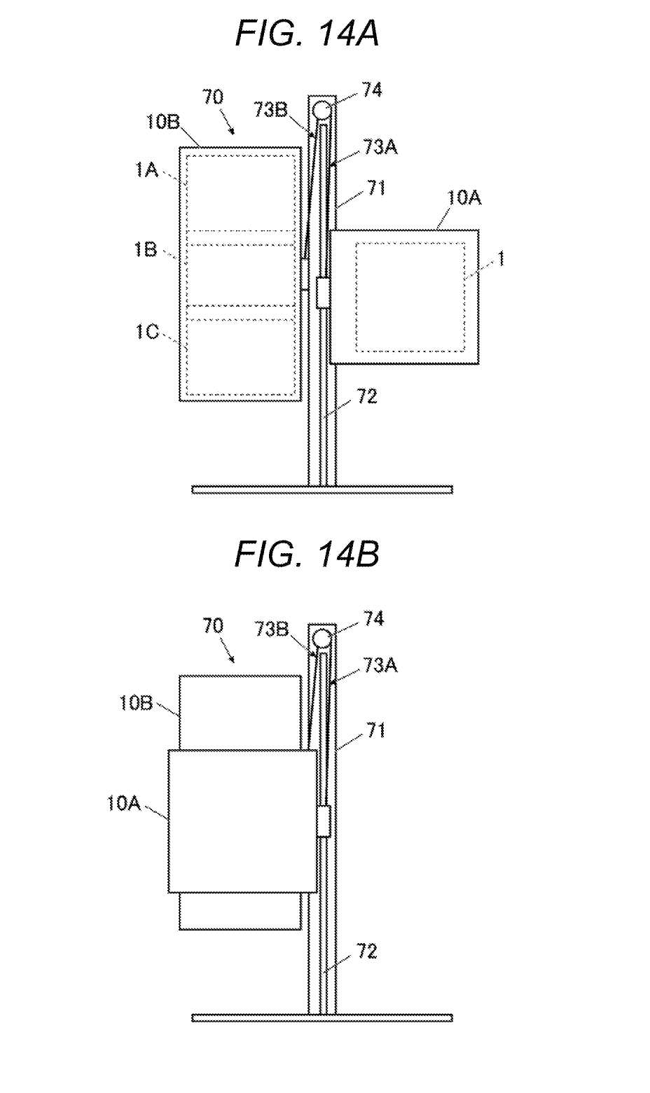

The position securing members 64 are designed to secure the radiological imaging apparatuses 1a and 1b in such a manner that the radiological imaging apparatuses 1a and 1b are located in the same position with respect to the radiological imaging apparatus 1. Although the three radiological imaging apparatuses 1, 1a, and 1b are provided in the example illustrated in FIG. 13B, the number of radiological imaging apparatuses 1 placed in the imaging stand 60 for performing long-length imaging through “one-time shooting” is not limited to three. The same applies in the cases illustrated in FIGS. 14A and 14B, which will be described later.

In FIG. 13B, the radiological imaging apparatuses la and lb are located on the upper side (or the patient side) of the radiological imaging apparatus 1. An automatic exposure controller (AEC) (not shown) is often required on the upper side (or the patient side) of the radiological imaging apparatus 1. In the configuration shown in FIG. 13B, the shadow of the automatic exposure controller is not captured by the radiological imaging apparatuses 1a and 1b, and high-quality images can be obtained in a preferred manner.

With the radiological imaging apparatuses 1 being provided as shown in FIG. 13B, radiation is released from a radiation source 30 in advance, and a correction value v can be calculated from the obtained distribution of the radiation intensity I_{R} in the above described manner. Although not shown in FIGS. 13A and 13B, a grid G can be placed on the entire lower surface of the top panel 61. In this case, radiation is also released from the radiation source 30 in advance, and a correction value v* for a case where a grid is provided can be calculated from the obtained distribution of the radiation intensity I_{R} in the above described manner.

Although not shown in the drawings, an imaging stand for upright imaging can be formed in the same manner as in the case illustrated in FIGS. 13A and 13B, with the portion of the bucky 10 shown in FIGS. 13A and 13B being changed to a vertical structure.

In the case of an imaging stand for upright imaging, however, there might be a restriction on the size of the bucky 10, since there is normally the need to provide a hand pole (not shown) to be gripped to keep a balance by a patient in a wheelchair or a patient who cannot easily stand up, for example. In a case where regular imaging is performed with at least one radiological imaging apparatus 1, if the portion of the bucky 10 shown in FIG. 13A is formed as an upright structure as described above, the size of the bucky 10 might become too large.

In view of this, a bucky that houses a single radiological imaging apparatus 1 for regular imaging and a bucky that houses radiological imaging apparatuses 1 for long-length imaging can be formed independently of each other. FIGS. 14A and 14B show an example structure of an imaging stand for upright imaging. FIG. 14A illustrates a case where long-length imaging is performed with radiological imaging apparatuses. FIG. 14B illustrates a case where imaging is performed with a single radiological imaging apparatus. FIGS. 14A and 14B illustrate situations seen from the side of a radiation source 30 (not shown).

As shown in FIG. 14A, the imaging stand 70 in this example structure includes a bucky 10A that houses a single radiological imaging apparatus 1, and a bucky 10B that houses three radiological imaging apparatuses 1A, 1B, and 1C that are secured beforehand in respective positions. The bucky 10A is attached to a first support pillar 71, and the bucky 10B is attached to a second support pillar 72. The bucky 10A and the bucky 10B can slide in the vertical direction. Alternatively, the bucky 10A and the bucky 10B may be attached to the same support pillar. Also, each of the radiological imaging apparatuses 1 and 1A through 1C may be detachably installed in the bucky 10A or 10B.

The bucky 10A that houses at least one radiological imaging apparatus 1 can be rotated about the second support pillar 72 (or a single support pillar), to be located in front of the bucky 10B housing the radiological imaging apparatuses 1A through 1C or on the side of the radiation source 30 (see FIG. 14B). The bucky 10A can also be rotated in reverse direction, to be located on the opposite side of the first support pillar 71 from the bucky 10B (see FIG. 14A).

Although not illustrated in the drawings, in the case where long-length imaging is performed through “one-time shooting”, the bucky 10A of the imaging stand 70 is placed as shown in FIG. 14A, and the object H (not shown) is made to stand in front of the bucky 10B housing the radiological imaging apparatuses 1A through 1C. Long-length imaging is then performed in this arrangement. In a case where regular imaging is performed, on the other hand, the bucky 10A of the imaging stand 70 is placed as shown in FIG. 14B, and the object H stays in a predetermined posture in front of the bucky 10A housing the single radiological imaging apparatus 1. Imaging is then performed in this arrangement.

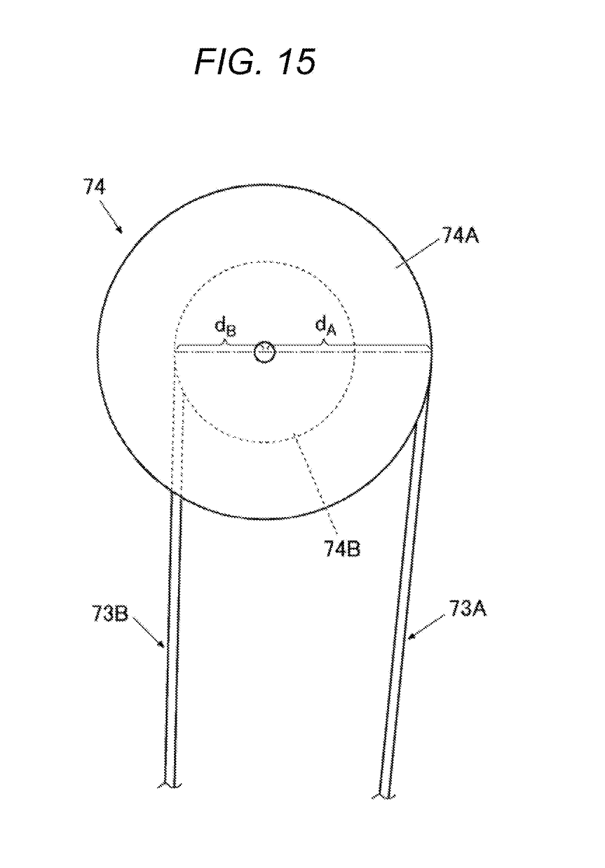

The bucky 10A and the bucky 10B are also attached to a pulley 74 on the first support pillar 71 via wires 73A and 73B, respectively. As shown in FIG. 15, the pulley 74 includes a pulley 74A having a large turning radius d_{A} and a pulley 74B having a small turning radius d_{B}. The pulley 74A and the pulley 74B are integrally formed around the same shaft. A wire 73A attached to the bucky 10A housing the single radiological imaging apparatus 1 is attached to the pulley 74A having the large turning radius d_{A}, and a wire 73B attached to the bucky 10B housing the radiological imaging apparatuses 1A through 1C is attached to the pulley 74B having the small turning radius d_{B}.

Therefore, the amount of torque the pulley 74 receives from the bucky 10A housing the single radiological imaging apparatus 1 is the same as the amount of torque the pulley 74 receives from the bucky 10B housing the radiological imaging apparatuses 1A through 1C, but the torque directions are the opposite of each other. In other words, the turning radii d_{A} and d_{B} of the pulley 74 are set so that such a situation is created. Accordingly, the bucky 10A housing the single radiological imaging apparatus 1 and the bucky 10B housing the radiological imaging apparatuses 1A through 1C are balanced by the pulley 74.

As a result, it becomes unnecessary to provide a counter weight to one or both of the bucky 10A and the bucky 10B. Since the bucky 10A and the bucky 10B are simultaneously moved up and down when the pulley 74 is rotated, only a single drive unit is required for rotating the pulley 74 to move the bucky 10A and the bucky 10B up and down. As there is no need to provide any counter weight, and only a single drive unit is required, the imaging stand 70 can be produced at low cost.

Also, the bucky 10A housing the single radiological imaging apparatus 1 needs to be moved up and down over a relatively large range so that the radiological imaging apparatus 1 is placed in an appropriate position with respect to the object H. However, the bucky 10B housing the radiological imaging apparatuses 1A through 1C does not need to be moved up and down over as large a range as the bucky 10A housing the single radiological imaging apparatus 1.

In this aspect, even if the bucky 10A housing the single radiological imaging apparatus 1 is moved up and down over a large range, the range over which the bucky 10B housing the radiological imaging apparatuses 1A through 1C moves up and down in synchronization with the movement of the bucky 10A does not become very large with the use of the structure shown in FIG. 15, for example. Accordingly, with the above described structure (see FIG. 15), the bucky 10A and the bucky 10B can be moved up and down in accordance with the respective operating characteristics of the bucky 10A and the bucky 10B.

Furthermore, with the above described structure, the position of a patient as the object H is substantially the same in front of the bucky 10B shown in FIGS. 14A and 14B both in the case where long-length imaging is performed through “one-time shooting” and in the case regular imaging is performed. The imaging stand 70 can be used, as long as there is a space that allows the bucky 10A to rotate about the second support pillar 72 (or a single support pillar). Accordingly, the above described imaging stand 70 can be used not only in a small imaging room but also in a small imaging room.

It should be understood that the present invention is not limited to the above described embodiments, and various changes may be made to them without departing from the scope of the invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. A radiological imaging system comprising:
a radiological imaging apparatus configured to generate image data in accordance with a dose of delivered radiation; and
a radiation source configured to deliver radiation to the radiological imaging apparatus,
wherein the radiation source is placed so that a tube axis of a rotating anode extends in a direction perpendicular to a body axis of an object being imaged.

2. The radiological imaging system according to claim 1,
wherein the radiation source is designed to be switched between a position in which the tube axis of the rotating anode extends in a direction parallel to the body axis of the object, and a position in which the tube axis of the rotating anode extends in a direction perpendicular to the body axis of the object.

3. The radiological imaging system according to claim 1, further comprising:
a mechanism configured to move the radiological imaging apparatus in a direction of the body axis of the object; and
a collimator provided between the radiation source and the radiological imaging apparatus, the collimator having an aperture and being movable in a direction parallel to the body axis of the object,
wherein radiation is released from the radiation source a plurality of times without change in release direction, and
long-length imaging is performed by moving the collimator in the direction parallel to the body axis of the object in synchronization with change in the position of the radiological imaging apparatus, so that radiation that is released from the radiation source and passes through the aperture is delivered to the radiological imaging apparatus being moved in the direction of the body axis of the object by the mechanism.

4. The radiological imaging system according to claim 1, further comprising
a plurality of radiological imaging apparatuses arranged in a direction of the body axis of the object, each of the plurality of radiological imaging apparatuses being the same as the radiological imaging apparatus,
wherein long-length imaging is performed by releasing radiation simultaneously to the plurality of radiological imaging apparatuses from the radiation source without change in release direction.

5. The radiological imaging system according to claim 1,
wherein the radiological imaging apparatus corrects the image data at least in the direction perpendicular to the body axis of the object based on a distribution of intensity of radiation released from the radiation source in the direction perpendicular to the body axis of the object.

6. The radiological imaging system according to claim 1, further comprising
an image processing device configured to correct the image data generated by the radiological imaging apparatus,
wherein the image processing device corrects the image data generated by the radiological imaging apparatus at least in the direction perpendicular to the body axis of the object based on a distribution of intensity of radiation released from the radiation source in the direction perpendicular to the body axis of the object.

7. The radiological imaging system according to claim 5,
wherein the radiological imaging apparatus or the image processing device calculates a correction value for each position with respect to the radiation source based on a distribution of intensity of radiation released from the radiation source in the direction perpendicular to the body axis of the object, and corrects the image data at least in the direction perpendicular to the body axis of the object based on the correction value, the distribution of intensity of radiation being acquired in advance.

8. The radiological imaging system according to claim 7,
wherein the correction value is obtained in each of a case where a grid is attached to the radiological imaging apparatus and a case where any grid is not attached to the radiological imaging apparatus.
